(19) United States
(12) Patent Application Publication
(10) Pub. No.: US 2024/0079091 A1
(43) Pub. Date: Mar. 7, 2024

(54) METHODS AND SYSTEMS FOR PHOSPHORMER MODEL EVALUATION

(71) Applicant: University of Georgia Research Foundation, Inc., Athens, GA (US)

(72) Inventors: Natarajan Kannan, Athens, GA (US); Zhongliang Zhou, Athens, GA (US); Sheng Li, Athens, GA (US); Wayland Yeung, Athens, GA (US)

(21) Appl. No.: 18/242,921

(22) Filed: Sep. 6, 2023

Related U.S. Application Data
(60) Provisional application No. 63/403,976, filed on Sep. 6, 2022.

(51) Int. Cl. G16B 40/00; G16B 30/20; G16B 45/00; G16B 50/30

(52) U.S. Cl. CPC G16B 40/00; G16B 30/20; G16B 45/00; G16B 50/30

(57) ABSTRACT

Various examples are provided related to phosphosite prediction. In one example, a system includes a computing device and an application for phosphosite prediction stored in memory. When executed, the application can cause the computing device to transform a protein sequence to a context-aware protein sequence by a Phosformer based transformer. The transformation can include predicting phosphorylation associations from the protein sequence based upon a trained Phosformer model and generating the context-aware protein sequence based upon the predicted phosphorylation associations, the context-aware protein sequence including a predicted phosphosite. The predicted phosphosite can be rendered for presentation to a user.

### CROSS REFERENCE TO RELATED APPLICATIONS

This application claims priority to, and the benefit of, co-pending U.S. provisional application entitled “Methods and System s for Phosphormer Model Evaluation” having Ser. No. 63/403,976, filed Sep. 6, 2022, which is hereby incorporated by reference in its entirety.

### BACKGROUND

Post-translational modifications (PTMs) facilitate diverse cellular signaling processes through the addition of specific functionalities to specific protein residues, allowing cells to appropriately respond to dynamic environments. Due to the importance of understanding specific kinase-substrate interactions, there has been an increasing interest in developing machine learning models for kinase-specific phosphorylation as opposed to general, non-kinase-specific phosphosite prediction. However, the prediction of kinase-specific phosphosites is more challenging due to the comparatively limited amount of experimentally validated data and an incomplete knowledge of sequence, structure and functional features associated with kinase-substrate interactions.

### SUMMARY

Aspects of the present disclosure are related to phosphosite prediction. In one aspect, among others, a system comprises a computing device comprising a processor and memory; and an application for phosphosite prediction comprising machine readable instructions stored in the memory that, when executed by the processor, cause the computing device to at least: obtain a protein sequence; transform the protein sequence to a context-aware protein sequence by a Phosformer based transformer, the transformation comprising: predicting phosphorylation associations from the protein sequence based upon a trained Phosformer model; and generating the context-aware protein sequence based upon the predicted phosphorylation associations, the context-aware protein sequence comprising a predicted phosphosite; and render the predicted phosphosite for presentation to a user. In one or more aspects, the Phosformer model can be pretrained based upon phosphorylation data. The phosphorylation data can comprise a plurality of kinase-substrate pairs. The kinase-substrate pairs can be generated from a plurality of experimental databases. In various aspects, the Phosformer based transformer can be trained using filtered protein sequencies. The filtered protein sequencies can be generated based upon a random mask. About 15 percent of domain segments can be randomly masked out. The filtered protein sequencies can comprise kinase-substrate sequences.

In another aspect, a method comprises obtaining, by at least one computing device, a protein sequence; transforming, by the at least one computing device, the protein sequence to a context-aware protein sequence by a Phosformer based transformer, where the transformation comprises: predicting phosphorylation associations from the protein sequence based upon a trained Phosformer model; and generating the context-aware protein sequence based upon the predicted phosphorylation associations, the context-aware protein sequence comprising a predicted phosphosite; and rendering the predicted phosphosite for presentation. In one or more aspects, the method can comprise pretraining the Phosformer model based upon phosphorylation data. The phosphorylation data can comprise a plurality of kinase-substrate pairs. In various aspects, the Phosformer based transformer can be trained using filtered protein sequencies. The filtered protein sequencies can be generated based upon a random mask. The filtered protein sequencies can comprise kinase-substrate sequences.

In another aspect, a non-transitory computer readable medium having a program, that when executed by processing circuitry, causes the processing circuitry to: obtain a protein sequence; transform the protein sequence to a context-aware protein sequence by a Phosformer based transformer, where the transformation comprises: predict phosphorylation associations from the protein sequence based upon a trained Phosformer model; and generate the context-aware protein sequence based upon the predicted phosphorylation associations, the context-aware protein sequence comprising a predicted phosphosite. In one or more aspects, the Phosformer model can be pretrained based upon phosphorylation data. The phosphorylation data can comprise a plurality of kinase-substrate pairs. In various aspects, the Phosformer based transformer can be trained using filtered protein sequencies. The filtered protein sequencies can be generated based upon a random mask. The filtered protein sequencies can comprise kinase-substrate sequences.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of a Phosformer model framework, in accordance with various embodiments of the present disclosure.

FIGS. 2A-2C illustrate an overview of the deep learning pipeline and architecture of the Phosformer, in accordance with various embodiments of the present disclosure.

FIGS. 3A and 3B illustrate performance of the Phosformer, in accordance with various embodiments of the present disclosure.

FIG. 4 is a table illustrating comparisons of the performance of the Phosformer to other methods, in accordance with various embodiments of the present disclosure.

FIGS. 5A-5C illustrates an example of substrate attention and explainability in substrate predictions, in accordance with various embodiments of the present disclosure.

FIGS. 6A-6C illustrates diversity of Phosformer prediction, in accordance with various embodiments of the present disclosure.

FIGS. 7A and 7B illustrate an example of the model architecture, in accordance with various embodiments of the present disclosure.

FIGS. 8A-8C illustrate examples of UMAP projections showing model learning process, in accordance with various embodiments of the present disclosure.

FIGS. 9A and 9B illustrate examples of SHAP values for two substrate peptide-kinase pairs, in accordance with various embodiments of the present disclosure.

FIG. 10 illustrates an example of a schematic block diagram of a computing device that can be utilized to execute a Phosformer model application, in accordance with various embodiments of the present disclosure.

### DETAILED DESCRIPTION

Disclosed herein are various examples related to phosphosite prediction. A Phosformer, a deep learning model which achieves a new state-of-the-art performance for kinase-specific phosphosite prediction is presented. Using a novel transformer-based architecture, the Phosformer generates biologically meaningful features in an unbiased and unsupervised manner. Because feature generation is integrated into the prediction model, Phosformer is highly amenable for high-throughput predictions. While the application of Phosformer in phosphorylation prediction is demonstrated, the model can be applied to predict any post-translational modification of interest such as, e.g., sulfation, glycosylation, ubiquitination, methylation, glycation, lipidation, nitrosylation, acetylation, proteolysis, hydroxylation to name but a few. Reference will now be made in detail to the description of the embodiments as illustrated in the drawings, wherein like reference numbers indicate like parts throughout the several views.

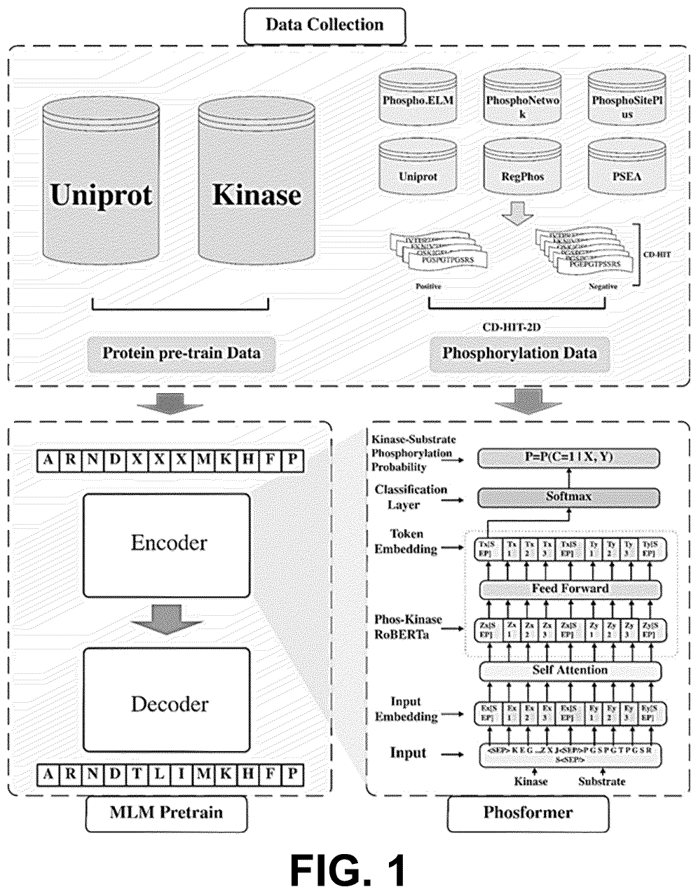

FIG. 1 illustrates an example of the Phosformer model framework. As illustrated in FIG. 1, the Phosformer model framework comprises data collection, mask language modeling (MLM) pretraining, and the Phosformer. The top panel illustrates an example of the data collection and preprocessing pipeline. The left bottom side panel illustrates an example of the mask language modeling procedure that utilizes unsupervised protein representations to encode peptides. The right bottom side panel illustrates an example of the framework for kinase-substrate phosphorylation prediction. Both kinase domain region and the corresponding phosphorylate peptide can be used as input.

Data collection and preprocessing. For the kinase-substrate data, data was extensively collected from 6 experimental databases including Phospho.ELM, PhosphoNetwork, PhosphoSitePlus, Uniprot, RegPhos and PSEA. After reducing redundancy, 21,955 experimentally verified phosphorylation sites with kinase labels were selected for model training. To construct a negative dataset, substrates with the same AA (while not annotated as phosphorylation sites) were chosen as the initial negative dataset. Following previous methods, CD-HIT 2D and CD-HIT were used to further reduce negative data redundancy. A random downsampler was used to ensure the negative to positive ratio was below 5. The impact of the size of substrates was also explored by grid searching different substrates lengths starting from 11 to a maximum of 41. For kinase protein, the kinase aligned domain region was used for training. The alignment was performed using Mapgaps.

For the general phosphorylation predictions, data from the aforementioned databases that don't have kinase annotations were used. Similarly, CD-HIT-2D and CD-HIT procedures were adopted to reduce data redundancy. The final datasets contained 16,970 positive and 16,480 negative positions. Additional details on the dataset are provided in Table 1 below.

TABLE 1
(statistic of datasets)

| Type | AA_Index | Positive | Negative |
|---|---|---|---|
| General Phosphorylation Data | S/T | 14298 | 12958 |
| | Y | 2672 | 3522 |
| Kinase-specific Phosphorylation | S/T | 14099 | 55290 |
| Data | S/T/Y | 5760 | 21967 |
| | Y | 2096 | 4514 |
| Uniprot-curated Validation Data | S/T | 1098 | 5360 |
| | Y | 303 | 1335 |

For general phosphorylation prediction, 14,298 S/T sites and 2,672 Y sites data were collected and used. For kinase-specific phosphorylation, 14,099 kinase substrate pairs were used. Additionally, hold-out validation data collected from Uniprot to perform models' comparison was included.

Phosformar model design. The Phosformer model is first pretrained on human kinase-related and human protein databases using mask language modeling objectiveness. The pretrained model has two advantages over previous methods. First, compared with methods that used predefined features or amino acids embeddings, the pretrained model can effectively represent amino acid sequence with contextual information. Second, compared with previously released protein language models, the mask language model is specially modified for phosphorylation prediction. Being 10 times smaller, the models learn better representations compared to models that trained on the entire protein database.

For the kinase-specific phosphorylation prediction task, concepts from natural language processing can be employed that treat the kinase as a query and phosphorylated substrates as answers. Using the query-retrieval framework enables all kinase predictors to be unified into one model that can map multiple queries to an answer and also give multiple answers to a query. Further, training one unified model promotes better feature sharing between different kinase-substrate relationships as many kinases do share common substrates and phosphorylation sites. In addition, in contrast to previous studies that either use machine learning methods or CNN/RNN based deep learning models, the model can reason arbitrary length sequences by using a self-attention mechanism. In contrast with some models that either explicitly input PPI information or use a submodule to learn kinase-substrate interaction. The presented model can learn such interaction implicitly during training with cross-attention calculation.

Since the model will take the kinase domain segments as input, it is likely that the model will overtrain those segments because of multiple occurrences. To avoid such issues and to make the model robust to different inputs, multiple ways were explored to augment the data. Initially, the bounds used to cut the domain randomly were shifted. Randomly masking out 15 percent of the kinase domain segment was also explored so that the model would not overwhelmingly rely on certain regions of the kinase domain for prediction. Finally, the two aforementioned data augmentation were combined with a random oversampling method to balance the dataset across different kinase families. From extensive evaluation, the model achieved further accuracy improvement with those data augmentations.

In addition to fine-tuning the model, a feature-based model that uses the embeddings generated from the language model as input features was considered. An XGBoost model was selected as a benchmark for its superiority in scalability. This model, which is denoted as PhosformerGeneral, was used for general phosphorylation prediction to test the effectiveness of learned representations.

Phosformer: Transformer-Based Model for Protein Kinase-Specific Phosphorylation Predictions

The Phosformer model is a transformer-based deep learning model for kinase-specific phosphosite prediction. Predictions are modeled as a context-dependent question answering task in which a probability of phosphorylation can be predicted given an arbitrary pair of unaligned kinase and substrate sequences. It is shown that Phosformer can implicitly learn evolutionary and biochemical features, removing the need for human-defined features. Further analysis has revealed that Phosformer can learn substrate specificity motifs and distinguish between functionally-distinct protein kinase families. Comparisons using a hold-out dataset indicate that the model significantly outperforms existing methods for kinase-specific phosphosite prediction, while solely using unaligned primary sequences as input.

The disclosed transformer-based deep learning model of Phosformer makes kinase-specific phosphosite predictions based on a pair of inputs—the unaligned kinase domain sequence and the substrate peptide sequence. The model is first trained to understand the “language of life” by masked language modeling (MLM) on diverse protein kinase and substrate sequences. This process teaches the model to generate its own features in an unsupervised fashion rather than relying on prior human-derived features. After MLM, the model is further trained to make kinase-specific phosphosite predictions using a question-answering framework. Further examination of the hidden layers suggests that Phosformer is capable of distinguishing functionally similar protein kinase families with shared substrate specificity determinants, thus allowing the model to predict novel kinase-substrate interactions. Phosformer demonstrates significant improvements compared to other state-of-the-art models, while also presenting a more generalizable and unified predictive framework.

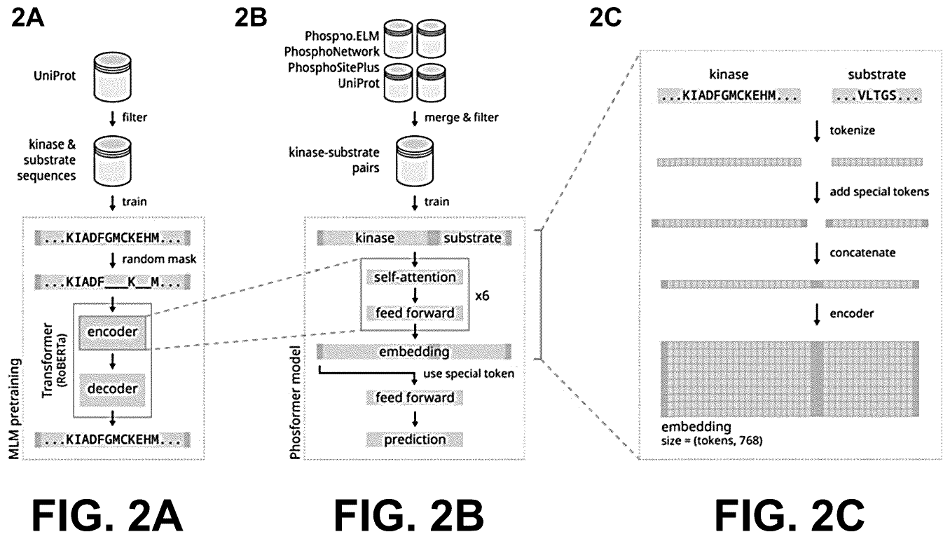

FIGS. 2A-2C illustrate an overview of the deep learning pipeline and architecture. In FIG. 2A, the transformer model can be trained to encode protein sequences using a dataset of protein kinase sequences from, e.g., UniProt proteomes. The bottom panel depicts masked language modeling (MLM), an unsupervised procedure for learning context-aware protein sequence embeddings. In FIG. 2B, the dataset of kinase-substrate pairs can be curated and filtered from four different databases. Using this dataset, a model, Phosformer, can be trained for predicting kinase substrate phosphorylation associations. The Phosformer architecture can be built from the pre-trained encoder comprising six attention units, which feed into a fully-connected feed-forward layer, followed by the final prediction layer. This feed-forward layer specially takes the first special token of the embedding vector. In FIG. 2C, kinase-substrate embeddings can be generated from the protein kinase domain sequence and a corresponding substrate peptide sequence with the phosphosite in the center position. The size of the resulting kinase-substrate embedding vector has a size corresponding to the number of tokens by 768.

Dataset curation and preprocessing. A dataset of experimentally validated kinase-specific phosphorylation sites was curated from a variety of databases: Phospho.ELM, PhosphoNetwork, and PhosphoSitePlus as shown in FIG. 2B. For each kinase-substrate pair, the kinase was represented as the unaligned protein sequence of the kinase domain, while the substrates were represented as 11-mer peptides, centered on the phosphorylative residue: serine (S), threonine (T), or tyrosine (Y). If the phosphorylation sites occurred within five residues of the N or Ctermini, the peptide was padded with “X” to ensure that all peptides have equal length and that the phosphosite occurs in the center position.

After curating the positive set, a negative dataset was defined using S, T, or Y sites in all substrate proteins which lacked phosphosite annotations. To reduce potential false negatives, the negative set was further filtered. This also reduces the overwhelming quantity of negative examples and avoids potential issues related to data imbalance. Following a previously defined strategy, any negative examples that were at least 40% similar to any positive example were removed using CD-HIT 2D, then the remaining examples were further filtered using 40% sequence similarity cutoff using CD-HIT. Finally, a random downsampler was applied to ensure that the ratio of negative to positive examples was below 5 to 1. The final dataset contained 25,688 positive and 109,210 negative sites spanning 800 unique protein kinases from human and other model organisms. Table 2 shows the statistics for the filtered dataset of kinase-specific phosphorylation sites curated from four databases. The examples are stratified by serine-threonine kinases (S/T) and tyrosine kinases (Y).

TABLE 2

| Group | Positive | Negative |
|---|---|---|
| Atypical | 1,020 | 5,100 |
| eLK | 5 | 25 |
| AGC | 6,080 | 24,142 |
| CAMK | 2,538 | 12,672 |
| CK1 | 552 | 2,744 |
| CMGC | 8,217 | 30,097 |
| Other | 2,202 | 10,806 |
| STE | 901 | 4,490 |
| TKL | 617 | 3,028 |
| TK | 3,556 | 16,106 |

The final dataset was divided into non-overlapping training, validations, and testing with a 70:15:15 ratio for all unique protein kinases with more than 50 positive examples, while protein kinases with less than 50 positive examples were limited to the training dataset. A total of 110 unique protein kinases were included in the testing and validation sets. The training dataset was used for model training and hyperparameter searching, while the validation dataset was used for early stopping. The testing set was used as the final benchmark for evaluation and comparisons against other models.

Masked language modeling pre-training. Protein sequence embeddings are numerical matrices that represent protein sequences. Embedding vectors are highly descriptive in that they can encode contextual information. It has been shown that sequence embeddings may innately encode structural, functional, and evolutionary information.

A transformer model with 12 attention heads, comprising a stack of 6 encoder layers followed by 6 decoder layers, was pre-trained. Protein sequences were encoded by a series of tokens, each representing one amino acid. The model was trained using the Mask Language Modeling (MLM) objective on kinase domain sequences with a mask ratio of 15%. The MLM procedure randomly replaces amino acid tokens with mask tokens in the input, then aims to restore the original amino acid tokens in the output. This can be formalized as:

$${L}_{MLM} = ( {X}_{\prod } \vert {X}_{- \prod } ) = \frac{1}{K} {\sum }_{k = 1}^{K} log p ( {X}_{{\prod }_{k}} \vert {X}_{- \prod } ; θ ) \qquad ( 1 )$$

where X_{Π} is the set of masked tokens in input, X_{-Π} is the set of unmasked tokens, K is the number of masked tokens in the input, and the indexes of the masked tokens in the input as Π=v₁, v₂, v₁, . . . , v_{K}.

The error was calculated by cross-entropy loss for back propagation during training. The model was trained for 50 epochs on 8 NVIDIA A5000s with a batch size of 48 per device. After MLM, the encoder was further used for fine-tuning phosphorylation prediction. The Phosformer model includes the pre-trained encoder followed by a fully connected layer leading to the final classification layer for phosphorylation prediction in FIG. 2B.

Modeling kinase-specific phosphorylation as a question answering task. The prediction of kinase-specific phosphorylation was modeled as a context-based question answering (QA) task. Typically used in the field of natural language processing, QA models take a “question” and “context” vectors and output an “answer” vector. Under this framework, the kinase sequence is the “question”, the substrate sequence is the “context”, and the probability of phosphorylation is the “answer”. This framework enables a unified model to be created for mapping diverse kinases to diverse substrates. Furthermore, the model is capable of understanding unaligned kinase sequences of arbitrary length and may implicitly learn kinase-substrate relationships through cross-attention calculations. In comparison, many existing models for phosphorylation prediction utilize CNN/RN N architectures, which explicitly encode amino acid or protein features, and are limited to fixed-sized aligned sequences. These limitations prohibit those models from effectively modeling long-range interactions which are crucial for biological inference.

A kinase and substrate sequence pair were encoded as a kinase-substrate embedding—analogous to the question-context embedding—which is subsequently used for predicting kinase-specific phosphorylation as shown in FIG. 2C. The kinase-substrate embedding comprises (1) a special token marking the start of the kinase domain, (2) tokens representing each residue of the kinase domain, (3) a special token marking the end of the kinase domain, (4) a special token marking the start of the substrate, (5) tokens representing each residue of the substrate, and (6) a special token marking the end of the substrate. Each token is represented by a vector of size 768.

Data augmentation strategy. Data augmentation strategies help produce robust models and avoid overfitting by increasing the amount of training data via meaningful modifications to the existing data. In protein research, since the labeled data are often sparse and unbalanced across groups, data augmentation is especially important for effectively training the neural network. Here, three data augmentation methods were defined and multiple combinations of these methods were used in the following experiments:
Shift: Randomly expand the boundaries of the kinase domain sequence by up to 10 residues. This will occasionally provide more sequence context for the regions flanking the kinase domain.
Mask: Randomly replace 15% of the kinase domain sequence with “X” which represents an uncertain amino acid residue. This encourages the model to consider the entire kinase sequence and discourages overly relying on a small subset of highly-informative residue positions.
Resample: Randomly resample protein kinase families with comparatively fewer training examples to balance the dataset. This strategy is only used in combination with random shift and mask to prevent duplicate training examples.

Performance evaluation. To fairly assess the proposed model on kinase-specific phosphorylation site prediction, evaluation metrics including the sensitivity, specificity, and Matthew's correlation coefficient (MCC) were adopted. These are defined as:

$$\begin{matrix}Sensitivity = \frac{TP}{TP + FN} & ( 2 ) \\ Specificity = \frac{TN}{TN + FP} & ( 3 ) \\ MCC = \frac{TP \times TN - FP \times FN}{\sqrt{( TP + FN ) ( TP + FP ) ( TN + FN ) ( TN + FP )}} & ( 4 )\end{matrix}$$

where TP, TN, FP, and FN stand for true positive, true negative, false positive, and false negative respectively. Sensitivity refers to the probability of a positive test, conditioned on truly being positive while specificity refers to the probability of a negative test, conditioned on truly being negative. To take both sensitivity and specificity into account, the widely applied area under the ROC curve (AUC ROC) was also used to assess the prediction. Higher AUC ROC scores indicate a better model. In addition, to offset the impact of an imbalanced dataset, MCC was used to quantify the performance between models.

Evaluation of protein-specific data augmentation strategy. The Phosformer architecture was trained using various combinations of data augmentation methods on the training dataset and each model was evaluated on the testing dataset. In addition to the unmodified testing set, the models were also benchmarked on an augmented version of the same testing set in which the boundaries of the kinase domains were independently shifted by up to three residues—simulating a more realistic use case with a user-derived variation. Overall results show that “shift+mask+resample” provides the most robust model, achieving an AUC-ROC of 0.929 on the unmodified testing set and 0.936 on the augmented testing set.

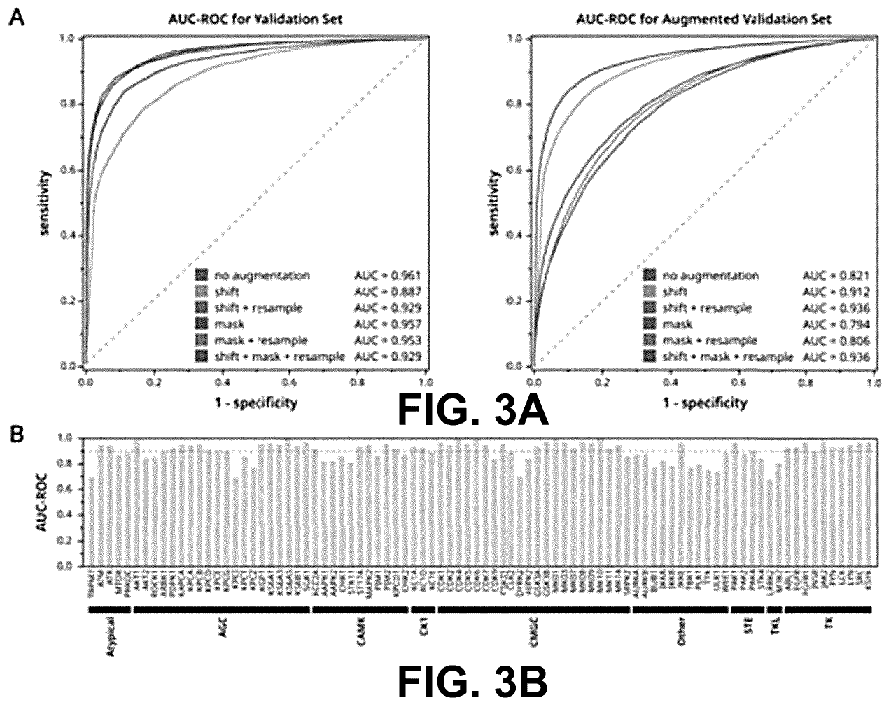

The Phosformer model's performance was further evaluated on kinase-specific predictions in the testing set. The model achieves an average AUC-ROC of 0.900 across 110 unique protein kinases and performs well in predicting substrates across a wide range of protein kinases shown in FIGS. 3A and 3B. The performance of the Phosformer model was measured with various data augmentation procedures. The performance is shown in FIG. 3A using AUC-ROC curves for the predictions of S/T phosphorylation sites (left) and Y phosphorylation sites (right). AUC values are provided at the bottom-right of each graph. In FIG. 3B, a bar plot shows the performance of Phosformer [shift+mask+resample] on kinase-specific prediction. Performance is shown along the y-axis, quantified by AUC-ROC. The dotted line shows the average AUC-ROC for all 110 unique kinases in the testing set. The gene names of the 88 human kinases are listed across the x-axis, stratified by group. The performance is particularly high for kinases with more training data such as CMGC and AGC and relatively modest for kinases with limited training datasets such as the STE, TKL, and Others groups. The overall performance across diverse phylogenetic groups suggests that the unified framework allows the Phosformer model to learn generalized features for predicting kinase-specific phosphorylation.

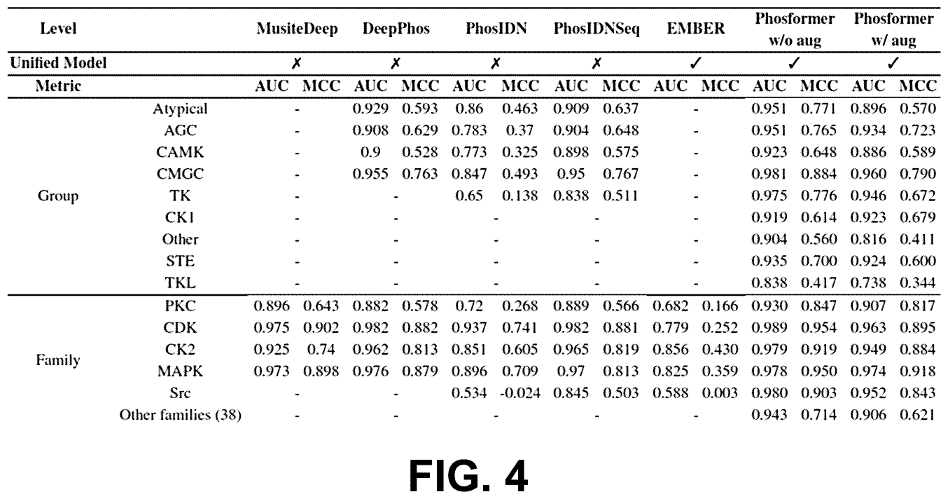

Comparison with existing methods. The performance of Phosformer was compared against five recently published methods for phosphosite prediction using the testing dataset. The table of FIG. 4 compares the performance of Phosformer to recently published methods for kinase-specific substrate phosphorylation prediction: MusiteDeep, DeepPhos, PhosIDN, PhosIDNSeq and EMBER. Model performance was quantified by the average AUC-ROC score, and MCC score, stratified by kinase group and family. Empty fields indicate that the corresponding model is unable to make predictions for the corresponding kinase group or family. Additional features were added to satisfy the specific input requirements of the other methods. Competing methods generally rely on separate family and group-specific models which resulted in a limited ability to predict phosphosites for diverse kinases families if the corresponding model was not trained or was not available. For instance, group-level models were not available for MusiteDeep, while the TK and Src models were not accessible for DeepPhos.

In order to facilitate meaningful comparisons, performance for each protein family and group level was evaluated. Compared to models that are specifically trained for individual families or groups, the Phosformer model demonstrates superior performance in group-level predictions, also demonstrating superior performance at the family level shown in the table of FIG. 4. Compared to a proposed unified model, Phosformer demonstrates significantly better results across different families. Overall, Phosformer generalizes well across a diverse range of protein kinase families, benefitting from the unique advantages of training a single, unified model.

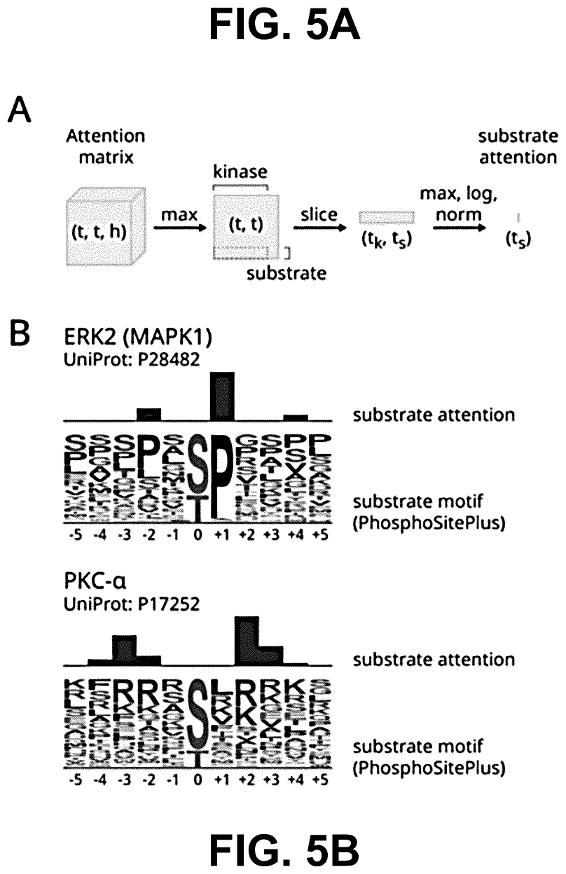

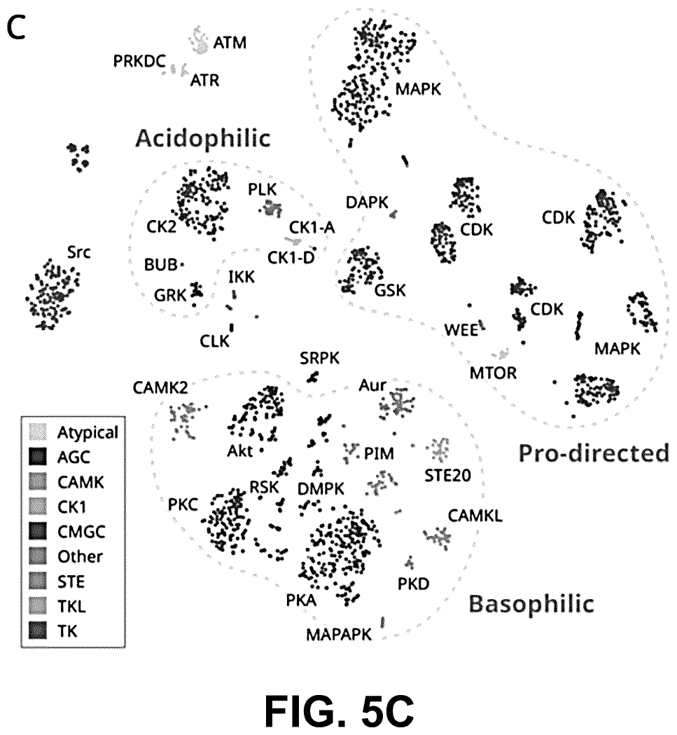

Kinase and substrate attention provides insights into specificity determinants. In order to investigate how the Phosformer model generates predictions, an explainability method was developed which quantifies the amount of attention directed toward each substrate residue position shown in FIG. 5A. The flow chart depicts the strategy for calculating substrate attention, based on the attention matrix of the final encoder layer. These calculations are derived from the final encoder layer. An in-depth analysis of two well-studied protein kinase families reveals that the Phosformer model is capable of learning substrate specificity motifs and identifying functionally-distinct protein kinase families.

FIG. 5B demonstrates the explainability in substrate predictions for human MAPK1 (top) and PKC-(bottom). The bar plot shows the average substrate attention from the testing set, while the sequence logo depicts experimentally determined substrate specificity motifs from PhosphoSitePlus. Substrate attention was calculated using the [mask+resample] model. The Phosformer predictions were investigated for MAPK1 substrates by determining the average substrate attention for positive examples in the testing set shown in FIG. 5B, top. Results indicate that the Phosformer directs more attention toward proline residues, particularly at the +1 and −2 positions. This is corroborated by previous studies which have shown that MAPK and CDK kinases are proline-directed with unique specificity determinants in the kinase domain that contribute to proline recognition. A similar analysis of PKC-substrates reveals that Phosformer directs more attention towards the +2 and +3 positions shown in FIG. 5B, bottom, which have previously been shown to be major specificity determinants of the PKC family kinases.

A t-SNE projection of kinase-substrate embeddings generated from Phosformer indicates that the model is capable of distinguishing between functionally-distinct protein kinase families. FIG. 5C shows a scatter plot depicting the manifold of kinase-substrate embeddings, illustrated as a two-dimensional t-SNE projection. The legend at the bottom-left lists the major protein kinase groups. Distinct clusters corresponding to protein kinase families are also labeled on the scatter plot. Overall, the manifold exhibits clusters corresponding to distinct protein kinase families where families from the same group tend to be closer together. The projection places AGC, CAMK, and STE kinases close together, all three of which tend to be basophilic. Proline-directed CMGC kinase families such as MAPK, CDK, and GSK are placed closer together, while the acidophilic CK2 family is placed farther away from the other CMGC kinases. Finally, the tyrosine kinases are placed at the edge, distinct from the serine/threonine kinases.

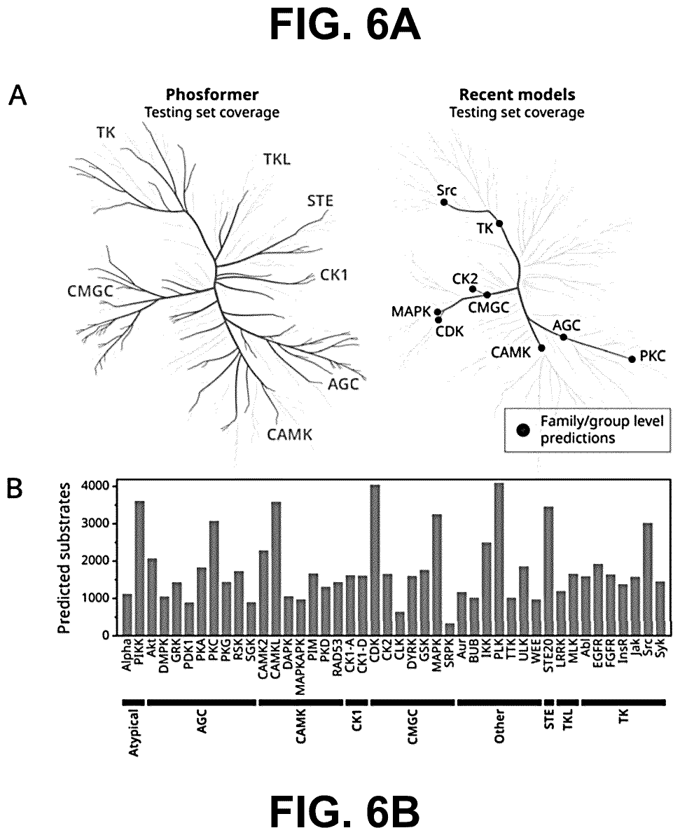

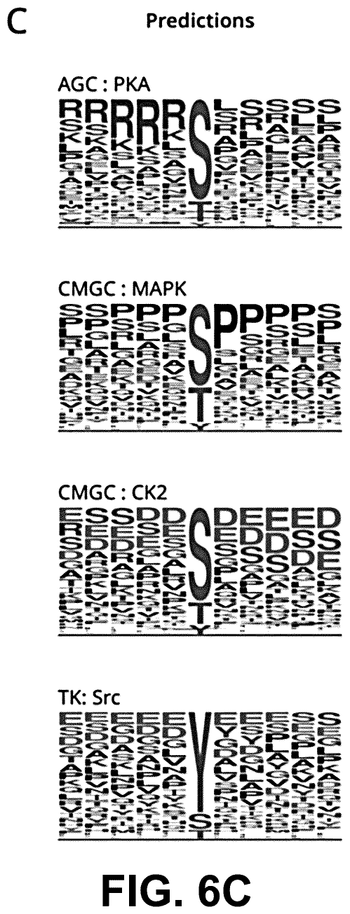

Kinase-specific phosphorylation predictions across the human proteome using Phosformer. The unique advantage of the Phosformer model is that it enables parallel predictions of all kinase-substrate pair relationships on individual sites. To demonstrate this application, kinase annotations were generated on all experimentally verified phosphosites in the human proteome. Predictions were filtered with a 0.95 probability threshold and 0.5 sequence similarity to focus on high-confidence and non-redundant predictions. The total number of predicted kinase-substrate pairs is 42,499 spanning across 9 groups shown in FIGS. 6A and 6B. Compared with the dataset that was curated during training, the predictions greatly expand the diversity of the kinase-substrate pair data on groups and individual kinases levels. This diversity enables further exploration of substrate phosphorylation determinants for individual kinases.

In FIG. 6A, two trees of the human protein kinome show the diversity of kinases upon which Phosformer can predict. The highlighted branch indicates the model can make inferences about certain individuals. Black dots denote the model can only make family or group level predictions. In FIG. 6B, a bar graph shows the number of predicted substrates for each protein kinase family.

Recently, the substrate sequence specificity of human serine/threonine kinases was profiled using synthetic peptide libraries, which provides an independent experimentally generated dataset to validate Phosformer predictions. In FIG. 6C, sequence logos show predictions for three diverse protein kinase families PKA, MAPK, CK2, and Src. A comparison reveals that Phosformer predictions of FIG. 6C are broadly similar to the substrate specificity motifs of a previous study. For example, both studies show a selective preference for arginine at the −2 and −3 positions in the PKA family substrate, a preference for proline at the +1 position for MAPK and CDK kinases, and an enrichment of serines and acidic residues for CK2 kinases. Overall, analysis of the predictions demonstrates that Phosformer is capable of recognizing substrate patterns that are distinct across diverse kinases by modeling the underlying kinase-substrate interactions through the attention mechanism.

Phosformer is a deep learning model which achieves state-of-the-art performance for kinase-specific phosphosite prediction. Using a novel transformer-based architecture, Phosformer can generate biologically meaningful features in an unbiased and unsupervised manner. Because feature generation is integrated into the prediction model, Phosformer is highly amenable for high-throughput predictions. All parts of the predictive pipeline can be written using the PyTorch library and benefit from multi-threading and GPU acceleration.

Unlike previous strategies, the Phosformer model uses a single unified model for the end-to-end prediction of kinase-specific phosphosites rather than using separate family-specific models. This allows the model to learn generalized features from well-studied kinases to improve the performance of less represented families. While previous strategies explicitly utilize human-curated features such as protein-protein interactions, protein kinase family classification, and sequence similarity, Phosformer utilizes a more unsupervised approach by allowing the transformer architecture to generate its own features based solely on primary, unaligned sequences. Analysis of the trained model revealed that Phosformer can learn substrate specificity motifs and distinguish between functionally-distinct protein kinase families—acidophilic, basophilic, or proline-directed. This ability is corroborated by previous results which have shown that transformer protein language models are unsupervised learners for biochemical, structural, and evolutionary features. The Phosformer model utilized the kinase domain sequence and substrate peptide as inputs. Incorporating the full kinase and substrate sequences can provide additional contextual information and may result in improved predictions. Training the model with an experimentally validated negative dataset could also reduce false-positive rates and improve performance.

Protein Language Model Learning Mechanisms for Kinase-Specific Phosphorylation Prediction

Protein phosphorylation is a key biological process in which a protein kinase adds a phosphate group to a protein, thereby modifying its structure and function to regulate various cellular activities. Here, a multitask attention based neural network is introduced that utilizes a protein language model as the backbone, modified specifically to enhance kinase-specific phosphorylation prediction called Phosformer-ST. The model not only demonstrated superior performance compared to existing models, but also addressed the interpretability and generalization challenges commonly found in neural networks. Diving deep into the model's training and reasoning methodologies using explainable AI tools, it can be observed that the robust performance of the model stems from its exceptional ability to accurately discern kinase evolutionary and functional relationships without supervised training on such tasks.

Here, a multitask deep learning model is presented that has been trained on this dataset to predict kinase-specific Serine/Threonine (ST) phosphorylation. By accepting a 15-residue peptide and a kinase sequence pair as input, the model demonstrates state-of-the-art performance in recognizing substrate specificity motifs and associating different kinases with multiple motifs using a unique strategy based on negative evidence. By further examining the learning process of the model using XAI tools such as manifold visualization and SHAP, it was found that the model has developed a novel understanding of kinase-substrate relationships and a unique strategy for phosphosite prediction, considering substrate motif patterns and kinases' evolutionary and functional relationships. This work underscores the potential of deep learning models to shed light on complex biological processes and emphasizes the importance of synergy between machine learning and human insights in enhancing our understanding and strategies in computational biology.

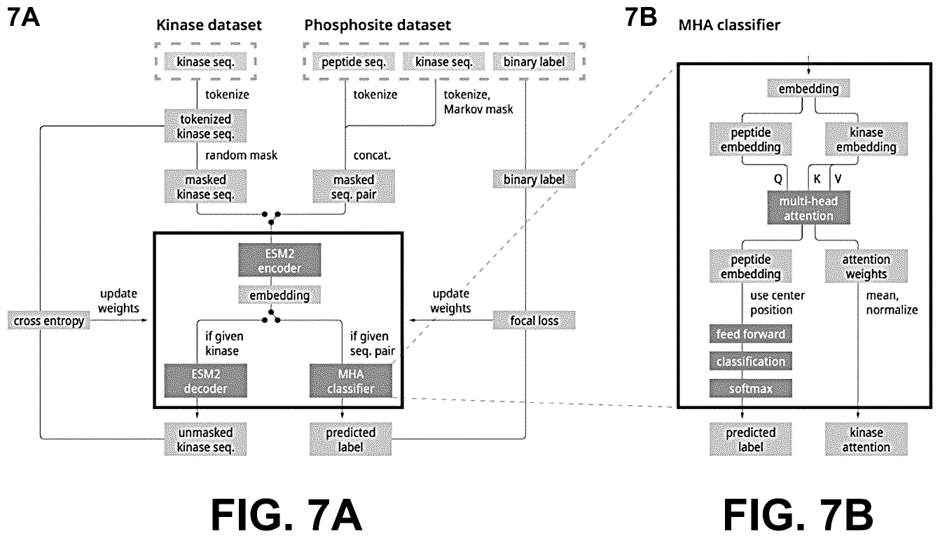

FIGS. 7A and 7B illustrate the model architecture. In FIG. 7A, a schematic shows multitask training on MLM (left) and kinase-specific phosphosite prediction (right), both sharing the ESM2 encoder. Single sequence inputs for MLM are directed to the ESM2 decoder, and paired inputs for phosphosite prediction go to the MHA classifier, both tasks updating the encoder weights. FIG. 7B shows a close-up of the MHA classifier that models kinase-peptide attention.

Dataset curation. In the previous study, 303 kinases' substrate specificity profiles were mapped to 89,784 experimentally-determined serine/threonine phosphosites, producing 27,204,552 unique kinase-peptide combinations. These combinations were scored from 0 to 1, with scores above 0.9 labeled as positive examples.

Out of the 303 kinases, 300 fitting the protein kinase-like structural fold were included, while three evolutionarily divergent ones were excluded. After removing duplicate and unmappable peptides, 86,043 phosphosite peptides remained. 884,203 unique serine/threonine sites with no phosphorylation evidence were also curated, allowing “hard” and “easy” negative examples to be defined.

Hamming distance based clustering was performed on the kinase sequences and peptides, resulting in 187 kinase clusters and 73,547 peptide clusters. The dataset was then built from these clusters, with positive and “hard negative” examples derived from pairs with scores above 0.9 and below 0.5, respectively, and “easy negatives” generated randomly from the non-phosphorylated substrates.

The dataset was divided into training, validation, and testing sets (60:20:20 ratio), ensuring an equal ratio of positive to negative examples in the latter two. The validation set was used for tuning, and the testing set served as a performance benchmark.

Model training. Utilizing the ESM2 encoder, which was pretrained from a large protein sequences corpus, the model was developed specially designed to predict kinase-specific phosphorylation. The model leverages two inputs, a 15-mer peptide sequence, and an unaligned kinase domain sequence, to predict the likelihood of the peptide's middle residue (S/T) being phosphorylated by the specified kinase.

The model employs multitask learning, simultaneously trained on two objectives, masked language modeling (MLM) and kinase-specific phosphosite prediction, through a shared encoder. The encoder outputs an embedding vector, processed by either a decoder (MLM) or a classifier (kinase-specific phosphosite prediction).

Kinase MLM: This objective learns the distinct features of kinase fold enzymes, trained on a curated dataset of 295,320 diverse kinase domain sequences from 18,832 organisms.

Kinase-specific phosphosite prediction: Given a peptide kinase pair, the model predicts whether the kinase can phosphorylate the peptide in vitro. Peptide-kinase embeddings are processed through an attention block, and the final prediction is generated from the embedding of the potential phosphosite, directed towards a binary classification layer.

Performance evaluation. Model performance was evaluated using two distinctive holdout testing sets to assess different aspects of model performance. Specifically, for the first testing dataset that contained a balanced ratio of positive and “hard negative” dataset, the AUC ROC score was used, and for the second testing dataset that only contains “easy negative” data, False Positive Ratio (FPR) was adopted for evaluation.

The model was compared against several most recent kinase specific phosphorylation prediction models including Deep-Phos, PhosIDN, EMBER, and MusiteDeep. The results show that the model performs better than all other compared models in terms of AUC ROC score and also makes fewer false predictions by lower FPR ratio.

Phosformer-ST learned kinase evolution and substrate binding specificity. The model's strategy was explored for understanding kinase-specific phosphosite prediction. Starting with a pre-trained ESM2 protein language model, which already has a basic understanding of biologically observed protein sequences, the model was further trained to predict kinase-specific phosphosites and language modeling for protein kinases.

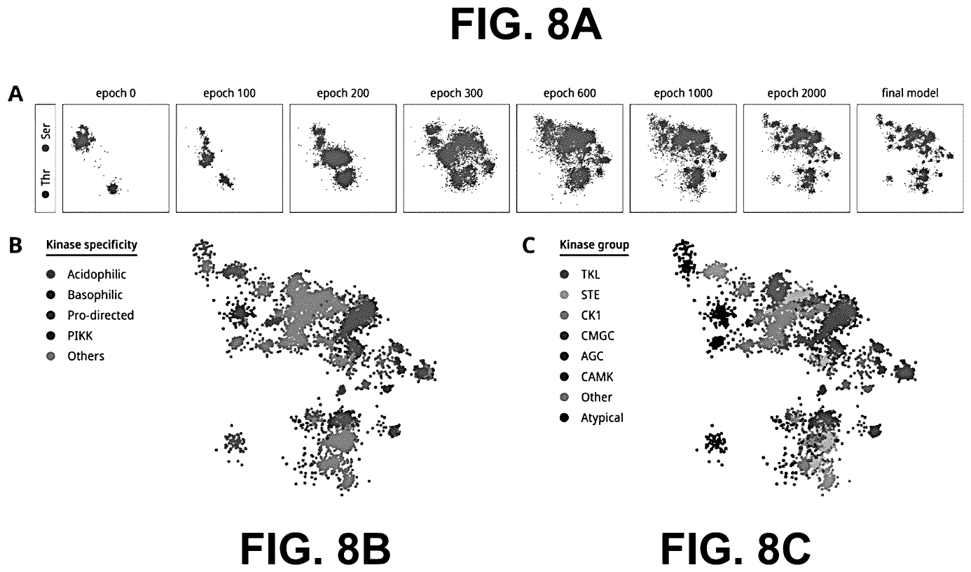

30 true positive instances were randomly selected from each kinase to examine how the model interprets this dataset of peptide-kinase pairs across various training epochs. The model's interpretation of a given pair is illustrated by a sequence embedding, specifically, the phosphosite residue embedding for comparative purposes. The relationships were visualized among embeddings from different training epochs using UMAP projections. FIGS. 8A-8C show UMAP projections illustrate the model's learning process. In FIG. 8A, a continuous series of UMAP projections show the relationships between embeddings—from the encoder—at various training epochs, indicated above each plot. Ser/Thr are colored red and green respectively. FIG. 8B shows the UMAP projections of the final model, re-colored by kinase specificity groups and FIG. 8C shows the UMAP projections of the final model, re-colored by evolutionary groups.

From FIG. 8A, it can be seen that at the initial stage (epoch 0), the pre-trained ESM2 model differentiates between Ser/Thr sites, with the additional variability within each cluster reflecting the transformer model's ability to encode sequence context information. Early in training, the model rapidly converges Ser/Thr sites into a single large supercluster (epoch 300). For the rest of the training, the model gradually fragments the supercluster into several smaller, distinct clusters, reflecting a complex organization based on substrate specificity motifs and evolutionarily-related kinase families (see FIGS. 8B and 8C).

Phosformer-ST develops a unique strategy for phosphosite prediction. The model's decision-making mechanism can be further examined by applying Shapley Additive Explanations (SHAP), a method to quantify the impact of each residue on the final prediction. Residues contribute positively or negatively, with the final prediction being their cumulative sum.

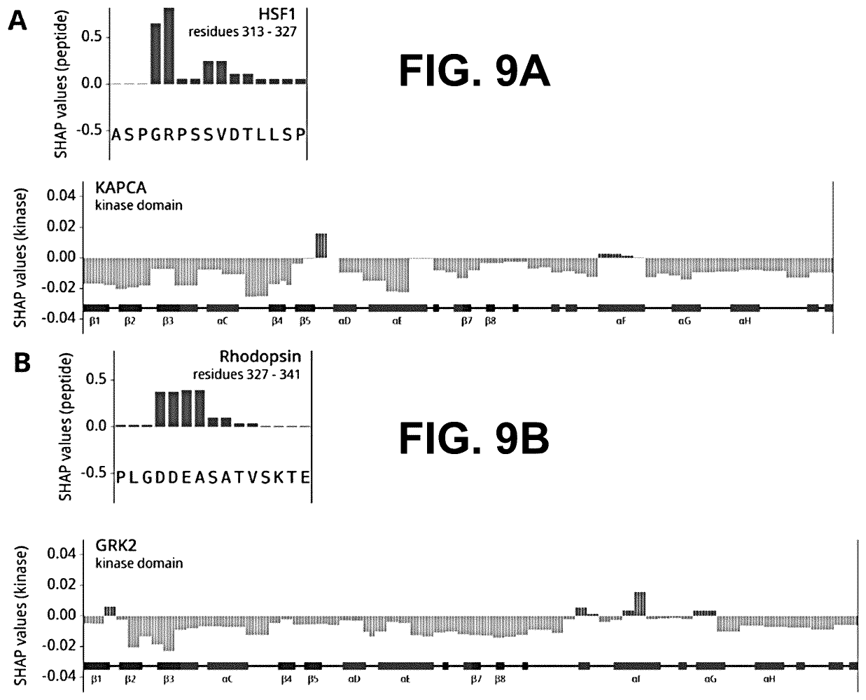

The analysis of SHAP values for two well-studied substrate-kinase pairs reveals that peptide sequence information generally contributes positively, while kinase sequence information contributes negatively, a trend observed throughout the dataset. FIGS. 9A and 9B includes bar plots showing SHAP values for two substrate peptide-kinase pairs (A) HSF1 and KAPCA and (B) Rhodopsin and GRK2, respectively. Across the y-axes, positive SHAP values are above zero and negative SHAP values are below zero. Across the x-axes, the peptide sequences are shown (top) as well as the secondary structure of the kinase (bottom). SHAP values were estimated using the partition method.

The negative contributions from kinase sequence information suggest that Phosformer-ST uses distinctive sequence motifs to exclude incompatible kinases. Specific residues known for substrate specificity determination show high positive contributions. For instance, in the PKA (KAPCA) substrate-kinase pair, a basophilic kinase, SHAP assigns high positive contribution to the −3 position R in the substrate (see FIG. 8A). Similarly, in the GRK2 substrate-kinase pair, an acidophilic kinase, acidic residues D and E in the substrate receive high positive contributions (see FIG. 8B).

Thus, Phosformer-ST effectively identifies substrate specificity motifs. Overall, Phosformer-ST's unique approach to kinase-specific phosphosite prediction leverages positive evidence from peptide phosphorylation and counterbalances it with the negative evidence of kinase incompatibility.

A novel kinase-substrate phosphorylation prediction model named Phosformer-ST has been introduced, which overcomes prior limitations and offers key innovations. The model adopts a comprehensive pre-processing protocol, utilizing large peptide array datasets and incorporating negative data, unlike previous mass-spectrometry-based methods that lacked substantial negative datasets. In vitro data was exclusively utilized, avoiding the methodological mix of in vivo and in vitro data seen in prior works.

The model implements a Markov masking protocol recognizing the co-evolving nature of protein residues, leading to a more robust model by avoiding over-reliance on proximal residues. Multitask learning is also employed for protein language models, enhancing model robustness compared to the single-task learning approach of the previous Phosformer model. A thorough explainability analysis was delivered, visualizing embeddings over training epochs, providing residue-level SHAP explanations, and examining kinase attention. This facilitates deeper comprehension of model predictions and underlying mechanisms. The model's potential for zero-shot learning broadens its application spectrum, enabling the prediction of kinase-substrate relationships even in the absence of prior information. This enhances the model's practical utility significantly.

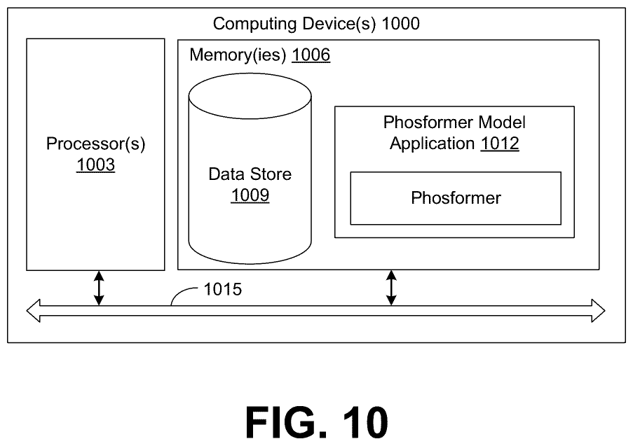

With reference to FIG. 10, shown is a schematic block diagram of a computing device 1000 that can be utilized to execute a phosformer model application 1012 for phosphosite prediction such as, e.g., kinase-specific phosphosite prediction. Each computing device 1000 includes at least one processor circuit, for example, having a processor 1003 and a memory 1006, both of which are coupled to a local interface 1015. To this end, each computing device 1000 may comprise, for example, at least one server computer or like device. In some embodiments, among others, the computing device 1000 may represent a mobile device (e.g., a smartphone, tablet, laptop computer, etc.). The local interface 1015 may comprise, for example, a data bus with an accompanying address/control bus or other bus structure as can be appreciated.

Stored in the memory 1006 are both data and several components that are executable by the processor 1003. In particular, stored in the memory 1006 and executable by the processor 1003 are the phosformer model application 1012 and potentially other applications. Also stored in the memory 1006 may be a data store 1009 and other data. In addition, an operating system may be stored in the memory 1006 and executable by the processor 1003.

It is understood that there may be other applications that are stored in the memory 1006 and are executable by the processor 1003 as can be appreciated. Where any component discussed herein is implemented in the form of software, any one of a number of programming languages may be employed such as, for example, C, C++, C #, Objective C, Java®, JavaScript®, Perl, PHP, Visual Basic®, Python®, Ruby, Flash®, or other programming languages.

A number of software components are stored in the memory 1006 and are executable by the processor 1003. In this respect, the term “executable” means a program file that is in a form that can ultimately be run by the processor 1003. Examples of executable programs may be, for example, a compiled program that can be translated into machine code in a format that can be loaded into a random access portion of the memory 1006 and run by the processor 1003, source code that may be expressed in proper format such as object code that is capable of being loaded into a random access portion of the memory 1006 and executed by the processor 1003, or source code that may be interpreted by another executable program to generate instructions in a random access portion of the memory 1006 to be executed by the processor 1003, etc. An executable program may be stored in any portion or component of the memory 1006 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1006 is defined herein as including both volatile and nonvolatile memory and data storage components. Volatile components are those that do not retain data values upon loss of power. Nonvolatile components are those that retain data upon a loss of power. Thus, the memory 1006 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1003 may represent multiple processors 1003 and/or multiple processor cores and the memory 1006 may represent multiple memories 1006 that operate in parallel processing circuits, respectively. In such a case, the local interface 1015 may be an appropriate network that facilitates communication between any two of the multiple processors 1003, between any processor 1003 and any of the memories 1006, or between any two of the memories 1006, etc. The local interface 1015 may comprise additional systems designed to coordinate this communication, including, for example, performing load balancing. The processor 1003 may be of electrical or of some other available construction.

Although the phosformer model application 1012 and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the phosformer model application 1012, that comprises software or code can be embodied in any non-transitory computer-readable medium for use by or in connection with an instruction execution system such as, for example, a processor 1003 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the phosformer model application 1012, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 1000, or in multiple computing devices in the same computing environment. Additionally, it is understood that terms such as “application,” “service,” “system,” “engine,” “module,” and so on may be interchangeable and are not intended to be limiting.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A system, comprising:
a computing device comprising a processor and memory; and
an application for phosphosite prediction comprising machine readable instructions stored in the memory that, when executed by the processor, cause the computing device to at least:
obtain a protein sequence;
transform the protein sequence to a context-aware protein sequence by a Phosformer based transformer, the transformation comprising:
predicting phosphorylation associations from the protein sequence based upon a trained Phosformer model; and
generating the context-aware protein sequence based upon the predicted phosphorylation associations, the context-aware protein sequence comprising a predicted phosphosite; and
render the predicted phosphosite for presentation to a user.

2. The system of claim 1, wherein the Phosformer model is pretrained based upon phosphorylation data.

3. The system of claim 2, wherein the phosphorylation data comprises a plurality of kinase-substrate pairs.

4. The system of claim 3, wherein the kinase-substrate pairs are generated from a plurality of experimental databases.

5. The system of claim 1, wherein the Phosformer based transformer is trained using filtered protein sequencies.

6. The system of claim 5, wherein the filtered protein sequencies are generated based upon a random mask.

7. The system of claim 6, wherein about 15 percent of domain segments are randomly masked out.

8. The system of claim 5, wherein the filtered protein sequencies comprise kinase-substrate sequences.

9. A method, comprising:
obtaining, by at least one computing device, a protein sequence;
transforming, by the at least one computing device, the protein sequence to a context-aware protein sequence by a Phosformer based transformer, where the transformation comprises:
predicting phosphorylation associations from the protein sequence based upon a trained Phosformer model; and
generating the context-aware protein sequence based upon the predicted phosphorylation associations, the context-aware protein sequence comprising a predicted phosphosite; and
rendering the predicted phosphosite for presentation.

10. The method of claim 9, comprising pretraining the Phosformer model based upon phosphorylation data.

11. The method of claim 10, wherein the phosphorylation data comprises a plurality of kinase-substrate pairs.

12. The method of claim 9, wherein the Phosformer based transformer is trained using filtered protein sequencies.

13. The method of claim 12, wherein the filtered protein sequencies are generated based upon a random mask.

14. The system of claim 12, wherein the filtered protein sequencies comprise kinase-substrate sequences.

15. A non-transitory computer readable medium having a program, that when executed by processing circuitry, causes the processing circuitry to:
obtain a protein sequence;
transform the protein sequence to a context-aware protein sequence by a Phosformer based transformer, where the transformation comprises:
predict phosphorylation associations from the protein sequence based upon a trained Phosformer model; and
generate the context-aware protein sequence based upon the predicted phosphorylation associations, the context-aware protein sequence comprising a predicted phosphosite.

16. The non-transitory computer readable medium of claim 15, wherein the Phosformer model is pretrained based upon phosphorylation data.

17. The non-transitory computer readable medium of claim 16, wherein the phosphorylation data comprises a plurality of kinase-substrate pairs.

18. The non-transitory computer readable medium of claim 15, wherein the Phosformer based transformer is trained using filtered protein sequencies.

19. The non-transitory computer readable medium of claim 18, wherein the filtered protein sequencies are generated based upon a random mask.

20. The non-transitory computer readable medium of claim 18, wherein the filtered protein sequencies comprise kinase-substrate sequences.
